(19) United States
(12) Patent Application Publication
(10) Pub. No.: US 2008/0045604 A1
(43) Pub. Date: Feb. 21, 2008

(54) COMPOSITION AND METHOD FOR FACILITATING THE HEALING OF NON-HEALING AND SLOW-HEALING WOUNDS AND ULCERATIONS

(76) Inventors: Lindsey Berkson, Austin, TX (US); Kenneth G. Burton, Sunrise Beach, TX (US)

Correspondence Address: Herbert Joe, 1801 Crystal Dr., #1009, Arlington, TX, 22202, US

(21) Appl. No.: 11/924,871

(22) Filed: Oct. 26, 2007

Related U.S. Application Data
(63) Continuation-in-part of application No. 10/992,636, filed on Nov. 17, 2004.
(63) Continuation-in-part of application No. 10/992,623, filed on Nov. 17, 2004.
(60) Provisional application No. 60/863,170, filed on Oct. 27, 2006.

(51) Int. Cl. A61K 31/04; A61K 31/12; A61P 17/00

(52) U.S. Cl. 514/679; 514/740

(57) ABSTRACT

The invention is of a composition for treatment of non-healing and slow-healing wounds and ulcerations, and for treating and preventing peripheral neuropathy. The composition is primarily characterized by a combination of nitroglycerin and turmeric. Preferred embodiments comprise emollient cream, mineral oil, and zinc oxide.

### CITATION TO PRIOR APPLICATION

This is a non-provisional conversion of U.S. Provisional Patent Application No. 60/863,170, filed 27 Oct. 2006. This is also a CONTINUATION-IN-PART with respect to U.S. application Ser. No. 10/992,636, filed 17 Nov. 2004 (17.11.2004) and of U.S. application Ser. No. 10/992,623, filed 17 Nov. 2004 (17.11.2004), from both of which priority is claimed under 35 U.S.C. §120 and under provisions of the Paris Convention and of the Patent Cooperation Treaty.

### BACKGROUND OF THE INVENTION

1. Field of The Invention

The present invention generally relates to an improved composition for treatment of skin conditions. More specifically, the present invention relates to a new wound management protocol, especially effective for highly compromised patients.

2. Background Information

Wound healing is a highly complex process, involving multiple, coordinated interactions of multiple factors and agents. Poor wound healing in diabetics, patients on dialysis, elderly in nursing homes, paralyzed/confined patients in wheel chairs, and patients on hospice is believed to relate to circulatory impairment and its sequelae.

Increasing numbers of patients who are aging, have compromised vascularization, become hospitalized, or have diabetes, are prone to non-healing ulcers in the feet and lower extremities. Foot ulceration is one of the most challenging problems of patients with diabetes especially those requiring dialysis. Non-healing foot ulcers are a serious issue in diabetic patients, in patients undergoing renal replacement (hemo- and CAPD dialysis), and especially in the patient population that suffers with both situations (end-stage renal disease [ESRD] secondary to diabetic nephropathy). In non-healing lesions inflammation is out of control. With uncontrolled inflammation, there is poor wound healing and often pain in those patients whose nerves are still intact.

These patients presently have no answer for non-healing lesions and are prone to a high amputation rate. There is also a high fatality risk within the year following amputations. Clearly, there is a great medical need in an unmet medical niche.

Wound management for patients with end-stage renal disease (ESRD), those with diabetes and those with transplants, is fraught with failure. This is especially true once they develop a non-healing lesion in the presence of the requirement of dialysis or other disease processes which compromise the healing process. These types of non-healing wounds cause great suffering for patient and the family and great difficulty for the care givers.

Furthermore, non-traumatic lower limb amputation is a serious potential complication of non-healing lesions, especially in diabetics and dialysis patients, and even more so when a patient suffers with both (diabetic patients on dialysis). The rate of amputation among diabetic persons with ESRD is 10 times as great as the diabetic population (which is already high), and two-thirds of these patients die within two years of the first amputation.

Extensive research leads the present inventors to believe that the most vital elements for successful wound healing are:

1. Nitric Oxide;

2. Endothelial nitric oxide synthase (eNOS/iNOS) (enzyme to make NO from arginine which is in sufficient amounts in properly functioning endothelium and not in diabetic and other compromised patients);

3. L-arginine (arginine is needed in sufficient amounts in local tissues to be acted on by eNOS to make NO and is often deficient in diabetic and other compromised populations);

4. Peptide growth factors (especially transforming growth factor beta-TGF-beta);

5. A healthy blood supply (local and systemic);

6. Ability to form new blood supply (angiogenesis);

7. Elimination or absence of microbes causing infection/excessive inflammation;

8. Sufficient macrophages;

9. Normal levels of homocysteine; and

10. Turmeric.

In highly compromised populations, such as diabetics, dialysis patients, confined patients, and the chronically ill and elderly, many of these essential factors are missing, or are deleteriously deficient.

The present inventors believe that, when all the above factors are in proper balance, de facto debridement occurs, new cell island matrix flourish, pernicious microbes are minimized, and the three stages of wound healing (a healthy amount of inflammation, proliferation and remodeling) occur efficiently and rapidly.

Various treatments for ulcer-type skin conditions are known in the art, yet none addresses the totality of factors needed to adequately and successfully facilitate healing of that which would be described clinically as “non-healing” or “slow-healing” wounds and ulcerations.

Facts and statistics relating to non-healing and slow-healing wounds and ulcerations, and their underlying origins or propensities, are sobering.

Type II diabetes is one such factor, and is the most common form of the disease, accounting for 90 to 95 percent of all diabetes cases.

Throughout the world, the incidence of Type II diabetes is nearing epidemic proportions. Examination of current and expected diabetic trends (and the detrimental effects thereof) is helpful for grasping the tremendous need for the present invention.

By way of example, the Center for Disease Control and Prevention (“CDC”) reports an increase in the cases of diagnosed adult diabetes of 49% between 1990 and 2000. Furthermore, the CDC estimates that diabetes, both diagnosed and undiagnosed, affects approximately seventeen million Americans (or approximately 6.2% of the U.S. population).

Diabetes is a prevalent disease and an ever-growing domestic and international public health concern. The World Health Organization (WHO) estimates that approximately 150 million people are affected by diabetes, and these numbers are expected to only increase to an estimated 215 million people by 2010 and an estimated 300 million people by 2025. Worldwide, diabetes has a relatively high mortality rate. Diabetes is reportedly among the top five causes of death by disease in most countries. More likely, diabetes is even more deadly, as it is frequently under-reported on death certificates.

Importantly for present purposes—the occurrence of diabetes and skin ulcers and non-healing wounds and ulcerations is directly related. Accordingly, the sharp increase in the number of diabetes cases has led to an increase in the number of people affected by non-healing and slow-healing wounds and ulcerations.

By way of example, diabetics have a 15% chance of developing a foot ulcer during their lifetime. Of those diabetics that develop foot ulcers, approximately 20% will require amputation. (International J of Pharm Compounding 8(4) July/August 2004, 269). Such amputations are also increasing at an alarming rate. Between 1990 and 2000, the number of amputations resulting from foot ulcers increased by 26%. This trend is actually expected to increase. Foot ulcers cause approximately 85% of all diabetic amputation of the lower extremities (Emergency Medicine 36(8) Au 2004, 14-23). The number of such lower extremity amputations (LEAs) now exceeds 100,000 per year!

Recurring foot ulcers, and the amputations that may result, present a continuing problem on a national and global scale. In the event that an ulcer is successfully treated, it is more likely than not that the ulcer will reoccur. Recurrence rates associated with diabetic foot ulcers and the resulting LEAs are commonly as high as 50%—70% over a period of three to five years.

Those skilled in the art of wound treatment realize that the accepted standard of care is simply not working. Current medications and modes of treatment all too commonly fail to heal wounds and ulcers in compromised patients, and thereby fail to prevent such complications as infection and gangrene.

Overall, 50%—80% of patients having diabetic foot ulcers will heal within six months, assuming optimal management from a multi-disciplinary team. (Emergency Medicine 36(8) August, 2004, 14-23). However, all too common complications require hospitalization, painful and expensive surgery, a prolonged rehabilitation regimen (if rehabilitation is possible at all), and increased health care and/or taxpayer expense. With the incidence of ulcer recurrence as high as 70%, the healing of one ulcer is often rapidly followed by the development of a new one.

In view of the serious and often unconquerable consequences of diabetic ulcers alone, and in further view of the need to address non-healing and slow-healing wounds and ulcerations in other compromised patient populations, a great need exists for an improved treatment for non-healing and slow-healing wounds and ulcerations. There is more, however.

Another collateral or even related condition which afflicts many in the same patient populations as those discussed above with respect to non-healing and slow-healing wounds and ulcerations is that of peripheral neuropathy. Many of the same conditions and circumstances that contribute to non-healing and slow-healing wounds and ulcerations also contribute to, cause, or exacerbate peripheral neuropathy.

The direct and indirect cost and difficulty associated with care for the aforementioned issues are in the multi-billion dollar range. Known treatment regimens and the present standard of care rely heavily on the use of debridement and washing. In fact, with only conventional wound treatment regimens available, such steps are necessary, though debridement and washing typically results in scarring, non-closure of the wound, and/or recurrence.

It is medically, socially, politically and economically important that a more economic, less stressful wound management program be found to save many patients from continued suffering as well as amputation and death and reduce the cost to all payers. Despite all of the benefits from known treatment regimens, the present state of would care is woefully deficient, and leaves many patients with unending pain, extremity amputations, general disability, and even death.

### SUMMARY OF THE INVENTION

In view of the above, the general purpose of the present invention, which will be described subsequently in greater detail, is to provide a uniquely efficacious composition and associated method for the treatment of non-healing and slow-healing wounds and ulcerations, as well as peripheral neuropathy, which composition and method are neither anticipated, rendered obvious, suggested, nor even implied by any of the known compositions or methods of treatment, either alone or in any combination thereof.

Therefore, it is an object of the present invention to provide a composition for treatment of non-healing and slow-healing wounds and ulcerations.

It is another object of the present invention to provide a composition and method for treating (or preventing) peripheral neuropathy.

It is another object of the present invention to provide a composition and method of wound management that is a significant economic cost-savings compared to standard treatment regimens.

It is another object of the present invention to provide a composition and method of wound management that is significantly less stressful to the patient when compared to the stress of standard treatment regimens.

It is another object of the present invention to provide a composition and method of wound management that reduces the frequency and need of amputations.

It is another object of the present invention to provide a compound and associated method of use thereof in the treatment of wounds and ulcerations, which composition and use thereof obviates the need for debridement in wound and ulcer treatment.

It is another object of the present invention to provide a method for treatment of wounds and ulcerations, with particular efficacy for previously non-healing and slow-healing wounds and ulcerations.

It is another object of the present invention to provide a composition and associated method for treatment of non-healing and slow-healing wounds and ulcerations, at least in part, by effecting to an unprecedented level, improvement of circulation at the wound site and associated healing in otherwise non-responsive patients.

It is another object of the present invention to provide an improved composition for treatment of non-healing and slow-healing wounds and ulcerations that affects a high degree of pernicious microbe eradication, without requiring debridement or other pre-medication wound or ulcer manipulation or alteration.

It is another object of the present invention to provide an improved composition for treatment for non-healing and slow-healing wounds and ulcerations that facilitates peripheral nerve growth and regeneration.

It is yet another object of the present invention to provide an improved composition for treatment for non-healing and slow-healing wounds and ulcerations that affects and utilizes synergistic action of Nitroglycerin and turmeric.

In satisfaction of these and other related objects, the present invention is a new wound management protocol, especially effective for highly compromised patients, e.g., dialysis patients and patients with nephropathy secondary to diabetes who require dialysis or transplantation. It produces rapid healing in these patients who have failed to respond to standard care for a time range of weeks to years. It does so even in the face of multiple organism contamination. True infections still need, however, to be controlled by appropriate antibiotic intervention. This combination shrinks wounds and enhances re-granulation and re-epithelialization more effectively than prolonged standard care.

It consists of (1) a pharmaceutical ointment that is applied by the patient 2-3 times a day, and (2) a protocol for wound care that in essence “leaves the wound alone” and thus avoids washing and cleaning, and usually reduces or eliminates the need for regular debridement. Specifically, the patient applies 2 CC, or the amount to sufficiently cover the wound, without touching or disturbing the wound. The wound is then covered with a telfa pad. The wound is not to be debrided, cleaned, pulled at, washed, or exposed to water or soap. This protocol is applied twice a day.

The present invention, by way of a novel composition and associated methods of applying that composition, yields results that simply are not possible with any other known treatments.

The composition of the present invention comprises, principally as active ingredients, nitroglycerin and turmeric (or alternative curcumin-containing substance), which have been found by the present inventors to work synergistically to increase the absorption and distribution of each other, as well as to effect an unprecedented therapeutic result. In addition, however, other ingredients (such as the recited zinc and aloe vera constituents) are instrumental in maintaining the medicament on-site for treatment of wounds and ulcerations, such that the optimal therapeutic result is achieved.

In its preferred form (as presently believed, though variations in relative constituency will fall well within the scope of the present invention), the present composition comprises: nitroglycerin (Nitrobid) 2% ointment, an emollient cream base (PCCA emollient cream formulation), mineral oil (light 65-75 VIS liquid), turmeric, e.g., Curcumin Powder 95% or a curcumin-containing ingredient, such as a measure of turmeric sufficient to provide the desired measure of curcumin, or even a synthetic curcumin, Aloe Vera (freeze dried 200:1 powder), Zinc Oxide USP, and Arginine (L) USP (HCL powder). This composition is formed as the triturate powders and wet powders are combined with the mineral oil and then thoroughly mixed with emollient cream, QS'ed to the desired volume. The treatment protocol essentially involves (1) a pharmaceutical ointment that is applied by the patient 2-3 times a day, and (2) a protocol for wound care that in essence “leaves the wound alone” and thus avoids washing and cleaning, and usually reduces or eliminates the need for regular debridement. Specifically, the patient applies 2 CC, or the amount to sufficiently cover the wound, without touching or disturbing the wound. The wound is then covered with a telfa pad. The wound is not to be debrided, cleaned, pulled at, washed, or exposed to water or soap. This protocol is applied twice a day.

While the characteristics unique to this treatment protocol may at some point, at first appear to be subtle distinctions vis-a-vis existing prior art, these distinctions self-evidently yield a regimen that is different from any such known in the art and produces unexpected (and previously unachievable) results. For instance, the present method increases blood flow to nerves thereby increasing nerve growth. This expedites the growth of new island cells and allows skin to take root and grow. This appears to be a primary reason for the improved results not seen in any of the known treatment regimen for wound, ulcer or neuropathy conditions.

Further, Applicant has devised a compound that creates effective vasodilation of the underlying capillary bed in patients with compromised vascular function. The present invention eliminates the need for debridement while acting as an agent or substantially eradicating pernicious microbes.

While the synergistic action of the two primary constituents are likely not fully understood or explained at this time, the individual actions of the nitroglycerine and turmeric, and some aspects of their complimentary actions have been revealed, at least in part, through the present inventors' research and experimentation.

Nitrocilycerine

Nitroglycerin is a nitrate that has been approved by the FDA since 1938 to dilate blood vessels. It is frequently used in the management of angina pectoris. It was first synthesized in 1846 and first used in cardiac therapeutics by physicians since 1879.

Nitroglycerin is a nitric oxide (NO) donor. NO is a small radical that is pivotal for wound healing. Nitrates preferentially dilate blood vessels that are compromised. Nitroglycerin acts by donating nitric oxide, which relaxes the walls of blood vessels, especially large microvessels.

Non healing wounds especially in diabetic and dialysis patients are notoriously deficient in nitric oxide, as well as the specific enzymes that are involved in nitric oxide local production and in the substrate needed to make NO (the amino acid L-arginine).

Why is NO essential to enhance healing at the wound site?

1. NO improves angiogenesis (Angiogenesis is the process by which new blood vessels form by sprouting from pre-existing vessels);

2. NO improves inflammation (healthy inflammation is stage one of wound healing, but then it must be contained. NO does not allow it to intensify);

3. NO promotes cell proliferation;

4. NO enhances matrix deposition;

5. NO helps speeds up remodeling;

6. NO promotes re-epithelialization (Journal of Investigative Dermatology 1999 December; 13(6):1090-8) which enhances closure of the wound.

7. NO decrease viscosity by inhibiting platelet aggregation (diabetics and dialysis patients, and often ill and elderly, have excessive clotting of platelets which reduces circulation and healing) (Biological Pharmacology Bulletin 2003 August;26(8):1135-43); and

8. Nitrates enhance circulation by increasing red blood cell (erythrocyte) deformability. (International Journal of Clinical Pharmacologic Therapeutics 1998 July;36(7):398-402.)

Therefore, nitroglycerin:

1. Enhances arterial and venous vasodilation/circulation in large microvessels by donating NO;

2. Enhances erythrocyte deformability (enhances local circulation);

3. Decreases blood viscosity (improving local circulation); and

4. Improves tissue oxygen tension (tcpO2) (improving circulation and distribution of vital factors to all cells).

In the past, a problem with the use of nitroglycerin is that of headaches occurring in a significant number of patients. Among its many other positive attributes, the present composition, though utilizing nitroglycerin, produces no reported problems with headaches. The present inventors believe that this somehow relates to the presence of curcumin in the composition.

Another feature of nitroglycerine, as revealed through prior investigations for use in wound care, relates to the rapidity of its physiological reactions and ultimate dissipation. This characteristic has greatly limited nitroglycerine's efficacy, when used alone, in wound care, because, to put it plainly, it did not stick around long enough to effect significant would healing. Further still, prior attempts to use nitroglycerin alone, or in other combinations, revealed some patients' tendency to develop tolerance for the drug, with reduced efficacious results (such as were, to a limited degree, achieved in the first instance).

The combination of L-arginine and curcumin with nitroglycerine, as later detailed, provides a therapeutic compound which is more long acting, overcomes the tolerance issue, and also eliminates the headache side effects.

Turmeric

Turmeric, a representative of plant genus Curcuma, is a member of the ginger family Zingiberaceae. The active substance of turmeric is the polyphenol, yellow pigment curcumin, also known as C.I. 75300, or Natural Yellow 3. The systematic chemical name is (1E,6E)-1,7-bis(4-hydroxy-3-methoxyphenyl)-1,6-heptadiene-3,5-dione. It can exist at least in two tautomeric forms, keto and enol. The keto form is preferred in solid phase and the enol form in solution. Generally, curcumin exhibits anti-inflammatory anti-tumor, and antioxidant properties.

Turmeric (Curcuma longa, also called turmeric or kunyit in some Asian countries) is a spice commonly used in curries and other South Asian cuisine. Its active ingredient is curcumin. It is a significant ingredient in most commercial cury powders. Turmeric is also used to give a yellow color to some prepared mustards, canned chicken broth, and other foods (often as an inexpensive replacement for saffron).

The medicinal properties of the turmeric have for millennia been known to the ancient Indians and have been expounded in the Ayurvedic texts. In Ayurvedic medicine, which is a form of alternative medicine in use primarily in the Indian subcontinent, turmeric is thought to have many healthful properties. It is taken in some Asian countries as a dietary supplement, which allegedly helps with stomach problems and other ailments. It is popular as a tea in Okinawa, Japan. It is currently being investigated for possible benefits in Alzheimer's disease, cancer and liver disorders.

It is only in recent years that Western scientists have increasingly recognised the medicinal properties of turmeric. The reported medicinal properties of turmeric include nephro-protection, i.e., protection of kidneys, including transplanted kidneys. Since free radicals are abundant in chronic hyperglycemic states, and since there is high blood sugar in diabetics and dialysis patients, these conditions contribute to poor wound healing. Therefore, turmeric is extremely beneficial for diabetics and dialysis patients.

Other reported medicinal properties of turmeric include hepato-protection, i.e., protection of the liver, and powerful inhibition of pathogenic molds and dermatophytes and other significant pathogens, e.g., Giardia lamblia, pathogenic molds, and parasites, e.g., scabies. In comparison, curcumin has no antifungal activity.

Furthermore, according to a 2005 article in the Wall Street Journal titled “Common Indian Spice Stirs Hope,” research activity into curcumin, the active ingredient in turmeric, is exploding. Two hundred and fifty-six curcumin papers were published in the past year according to a search of the U.S. National Library of Medicine. Supplement sales have increased 35% from 2004, and the U.S. National Institutes of Health has four clinical trials underway to study curcumin treatment for pancreatic cancer, multiple myeloma, Alzheimer's, and colorectal cancer. It is also reported that turmeric can strengthen the blood-brain barrier against attacks that result from auto-immune diseases, like multiple sclerosis.

Investigations into the low incidence of colorectal cancer amongst ethnic groups with a large intake of curries compared with the indigenous population have suggested that some active ingredients of turmeric may have anti-cancer properties. Also, anti-tumoral effects against melanoma cells have been demonstrated. Second-stage trials of a turmeric-based drug as a possible treatment for cancer are currently underway. Furthermore, a recent study involving mice has shown that turmeric slows the spread of breast cancer into lungs and other body parts, but also enhances the effect of taxol in reducing metastasis of breast cancer. However, according to recent research results, the component curcumin causes degradation of the human protein p53. p53 is responsible for removing damaged cells that are likely to become tumors, suggesting curcumin could accelerate tumor development.

A 2004 UCLA-Veterans Affairs study involving genetically altered mice suggests that curcumin, the active ingredient in turmeric, might inhibit the accumulation of destructive beta amyloids in the brains of Alzheimer's disease patients and also break up existing plaques. “Curcumin has been used for thousands of years as a safe anti-inflammatory in a variety of ailments as part of Indian traditional medicine,” Gregory Cole, Professor of medicine and neurology at the David Geffen School of Medicine at UCLA said.

Another 2004 study conducted at Yale University involved oral administration of circumin to mice homozygous for the most common allele implicated in cystic fibrosis. Treatment with circumin restored physiologically-relevant levels of protein function. Recent studies have shown that turmeric can be effective in fighting a number of STDs, including chlamydia and gonorrhea.

Curry Pharmaceuticals, based in North Carolina, is studying the use of a curcumin cream for psoriasis treatment. Another company is already selling a cream based on curcumin called “Psoria-Gold,” which shows anecdotal promise of treating the disease.

Despite the diverse medicinal properties of turmeric and its long history, there are no reports of turmeric being used for or as part of a regimen for wound treatment, and particularly no known use in combination with nitroglycerin in the manner, and for the purposes stated herein.

As stated above, the active substance of turmeric is the polyphenol curcumin. Curcumin significantly accelerates wound healing as it enhances expression of TGF-beta1 and TGF-beta tllrc, both in normal and impaired healing wounds as demonstrated by immunohistochemistry (Biofactors 2002;16(1-2):29-43).

Curcumin increases eNos/iNOS within a wound. As discussed above, NO is a vital factor in wound healing and its production is regulated by inducible nitric oxide synthetase (iNOS) or also called endothelial NO (eNOS). During cutaneous wound repair, iNOS is induced in large quantities, in a normal non compromised patient. Decreased circulation, poor nutrition, deficient local enzymes, excess sugar within cells, insufficient arginine levels, all decrease the inducibility of iNOS. The presence of a functionally active iNOS is a crucial prerequisite for normal wound re-epithelialization (J Investigative Dermatology 1999 December;113(6):1090-8).

Curcumin enhances macrophage production (white blood cells that function to engulfs and kill foreign pathogens and microbes).

Curcumin augments vasodilation (Biological Pharmaceutical Bulletin 2003 August;26(8):1135-43).

Curcumin demonstrates anti-inflammatory action against mediators of inflammation (Phytomedicine 2005 June;12(6-7):445-52. Purified curcumin (more than other curcuminoids in turmeric: demethoxy- or bisdemethoxycurcumin) was found to reduce inflammatory mediators in vitro study.

In experimental animals, curcumin has been shown to be anti-diabetic, anti-inflammatory, cytotoxic and have antioxidant properties (Medical Science Monitor 2005 July″11 (7):BR228-234).

Curcumin helps fight off pathogens acting as a natural antibiotic. Curcumin has been found to be effective against bacteria, viruses and fungi.

Curcumin reduced mediators of inflammation from Neisseria gonorrhoeae-induced NF-kappaB signaling. Curcumin abolished the adherence of bacteria to cells in infection, emphasizing the high potential of curcumin as an anti-microbial compound without cytotoxic side effects (Biological Chemistry 2005 May;386(5):481-90).

Curcumin has antimicrobial action (Journal of Ethnopharmacology 2005 May 13;99(1):147-51 (Letters of Applied Microbiology 2004;39(5):401-6).

Curcumin has antifungal properties—100% phytotoxic against Lemma minor, Fitoterapia 2005 March;76(2):254-7.

Curcumin has anti-inflammatory, antioxidant, anticarcinogenic, antiviral and antiinfectious activities (Critical Reviews for Food Science and Nutrition 2004;44(2):97-111).

Curcumin shows antibacterial, anti-inflammatory, and antineoplastic activity (J Pharmacologic Pharmacology 2000 May:55(5):593-601.) A study assessed curcumin in vitro, and with skin absorption of Wistar rats.

Curcumin enhances angiogenesis (Journal of Physiologic Pharmacology 2005 March;56 Suppl 1:51-69. Angiogenesis is a prerequisite for wound healing. Curcumin is frequently studied for it's role in enhancing angiogenesis).

Curcumin has been shown in many animal studies to accelerate the repair of excision wounds in mice whole body exposed to various doses of gamma radiation (Journal of Surgical Research 2004 July;120(1):127-38.

Radiation disrupts normal response to injury and inhibits normal wound healing and increasing the time of healing. Rats pretreated with curcumin and then exposed to radiation, enhanced wound contraction, decreased healing time, increased synthesis of collagen, hexosamine, DNA, and NO, and improved fibroblast and vascular densities. (Surgical Research 2004 July;120(1):127-38).

Topical curcumin enhances cutaneous wound healing in rats and guinea pigs. Curcumin was effective both orally and topically in diabetic rats and mice. (Wound Repair Regeneration 1998 Mar.-Apr.'6(2):167-77.

Wounds of animals treated with curcumin showed earlier re-epithelialization, improved vascularization, increased migration of various cells including dermal myofibroblasts, fibroblasts, and macrophages into the wound bed, higher collagen content, and increase in transforming growth factor-beta1 confirmed by in situ hybridization and laser scan cytometry.

Transforming growth factor-beta1 enhances wound healing and curcumin increases the mRNA transcripts for this growth factor, demonstrating that it enhances the bodies own production of this growth factor, in a natural manner, which may be one mechanism of enhanced wound healing by curcumin (Wound Repair and Regeneration 1998 March-April;6(2):167-77).

With respect to safety, curcumin has been used in Phase 1 clinical trials. There was no treatment-related toxicity up to 8,000 mg/day and beyond that the only real problem being the bulky volume was unacceptable to the patients. This was taken orally, and our new drug is via the skin, which reduces systemic exposure significantly. (Anticancer Research 2001 July-August;21 (4B):2895-900).

With respect to the contribution of Arginine to the efficacy of the present composition, NO is generated in the local endothelium through the oxidation of the amino acid L-arginine. This occurs due to the action of the enzyme eNOS/iNOS.

NO causes vascular smooth muscle to relax causing vasodilation. In addition to being the substrate for eNOS, L-arginine facilitates the dimerization of two identical subunits (Diabetes Care. 2004 January;27(1):284-5), forming a homodimer. The enzyme is only active in the dimeric form. Under proper conditions, dimerization occurs rapidly, on a timescale of minutes. Once formed, the dimmer is stable (Journal of Biological Chemistry 2002 277:310200-31010)

NO is a small radical, formed directly from the amino acid L-arginine by three distinct isoforms of the enzyme nitric oxide synthase. The inducible isoform (iNOS) is synthesized in the early phase of wound healing by inflammatory cells, mainly macrophages. Curcumin enhances this production.

During the next proliferative phase, many cells participate in NO synthesis.

NO released through iNOS regulates collagen formation, cell proliferation (new growth of cells to fill in the wound), and wound contraction (for the wound to start growing smaller in the healing process). Arginine together with NO and curcumin administration promotes these aspects of wound healing.

Arginine regulates dimerization locally, and enhances wound strength and collagen deposition in rodents and humans (Wound Repair Regeneration 2003 May-June;11(3):198-203).

### DETAILED DESCRIPTION OF THE PREFERRED EMBODIMENT

In the preferred embodiment of the present medicament, and in the medicament upon which an associated method of treatment is based, the primary active ingredients are Nitroglycerin and Turmeric. In this preferred embodiment, the Nitroglycerin is in the form of two percent ointment (NITROBID), and the turmeric as curcumin in 95% powder form.

The preferred nitroglycerin-turmeric-based compositions of the present invention may be prepared according to the following disclosure and protocol, with variations appropriate to a desired scale or production as will be apparent to person skilled in the production of pharmaceutical preparations:

A. Constituents of Preferred Embodiment of Composition for remediation of dermal anomalies:

| Ingredients | Quantity | |
|---|---|---|
| Nitroglycerin (Nitrobid) 2% ointment | 10 | GM |
| Arginine (L) HCL Powder | 10 | GM |
| PCCA Emollient cream base | 100 | GM |
| Mineral Oil, Light 65-75 VIS liquid | 8.33 | ML |
| Curcumin 95% Powder | 0.07 | GM |
| Aloe Vera freeze dried 200:1 powder | 0.2 | GM |
| Zinc Oxide USP | 1 | GM |
| Total: | 124.5 | GM |

B. General Mixing Procedure of Preferred Embodiment of the Composition of the present invention:

1. Triturate powders and wet powders with mineral oil and mix thoroughly with emollient cream.

2. Q.S. to desired volume.

The formed composition may then be applied topically to any wound or ulceration (without debridement or washing), usually b.i.d. A treatment period between three and ten days is thought to be sufficient to heal the large majority of treated wounds. Extremely specific dosage is not now believed to be critical, and a “general coverage” of the wound site, generally such as one would apply a sun screen or other lotion, will produce the therapeutic result.

As with any multi-constituent composition, the recited, relative measures of constituent ingredients may be varied to some degree, without noticeably affecting the therapeutic effect of the present composition. For example, the present 0.2% Nitroglycerin formulation described above (2 mg per gram of medicament) is believed optimal, but 0.1 mg per gram to 50mg per gram is believed still safe (non-toxic) and efficacious, and, even if not optimal, still within the scope of the present invention, when used in combination with arginine and curcumin.

Likewise, the present formulation of arginine (0.1% or 100 mg per gram at 10%) is believed variable between 1 mg per gram up to 1000 mg per gram, with retained safety and efficacy. Curcumin is presently shown at an 0.08% strength (0.8 mg per gram), and a range of 0.1 mg per gram up to 50 mg per gram of medicament is believe efficacious and safe (and clearly within the scope of the present invention if efficaciously used as described, with the other active ingredients).

Furthermore, no evidence presently available would indicate that modest variations of relative constituency would defeat efficacy or safety, and the recited formulation appears to be roughly a center point of the most efficacious constituency ranges.

Therefore, although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

We claim:

1. A topical, medicinal composition comprising:
nitroglycerin; and
curcumin.

2. The composition of claim 1 wherein said curcumin is present as a constituent of turmeric.

3. A topical viscous medicament for topical application to wounds and ulcerations comprising:
nitroglycerin; and
curcumin;
said nitroglycerin and turmeric being suspended in a viscous carrier medium.

4. The composition of claim 3 wherein said curcumin is present as a constituent of turmeric.

5. The medicament of claim 3 wherein said nitroglycerin and turmeric are present substantially in the form of, and approximately in the proportions of ten parts 2% nitroglycerin viscous material, and approximately 1 part curcumin powder.

6. A topical medicament for treatment of therapeutically resistant wounds and ulcerations comprising:
Nitroglycerin
Arginine
Emollient cream base; and
Curcumin.

7. The composition of claim 6 wherein said curcumin is present as a constituent of turmeric.

8. A method for remediating therapeutically resistant wounds and ulcerations comprising the steps of:
selecting a topical medicament comprising nitroglycerin and curcumin;
applying said medicament topically to a portion of a human body having non-healing or slow-healing wound or ulceration.

9. The method of claim 8 wherein said curcumin of said medicament is present as a constituent of turmeric.

10. A method for facilitating blood circulation for the facilitation healing of wounds or ulcertation, or in the remediation of peripheral neuropathy comprising the steps of:
selecting a topical medicament comprising nitroglycerin and curcumin;
applying said medicament topically to a portion of a human body exhibiting symptoms of peripheral neuropathy.

11. The method of claim 10 wherein said curcumin of said medicament is present as a constituent of turmeric.
